(19) United States
(12) Patent Application Publication
(10) Pub. No.: US 2002/0151732 A1
(43) Pub. Date: Oct. 17, 2002

(54) Estrogen receptors

(76) Inventors: Claes Ohlsson, Vastra Frolunda (SE); Jan-Ake Gustafsson, Stockholm (SE); Margaret Warner, Stockholm (SE); Bo Angelin, Stockholm (SE)

Correspondence Address: Todd E. Garabedian, Ph.D., WIGGIN & DANA, One Century Tower, 265 Church Street, New Haven, CT, 06508-1832, US

(21) Appl. No.: 09/994,292

(22) Filed: Nov. 26, 2001

Related U.S. Application Data
(60) Provisional application No. 60/275,023, filed on Mar. 12, 2001.
(60) Provisional application No. 60/274,996, filed on Mar. 12, 2001.
(60) Provisional application No. 60/275,047, filed on Mar. 12, 2001.
(60) Provisional application No. 60/274,995, filed on Mar. 12, 2001.

(30) Foreign Application Priority Data
Jan. 5, 2001 (GB) 0100302.9
Jan. 5, 2001 (GB) 0100298.9
Jan. 5, 2001 (GB) 0100299.7
Jan. 5, 2001 (GB) 0100301.1
Mar. 6, 2001 (GB) 0105525.0

(51) Int. Cl. C07J 9/00; A61K 31/56

(52) U.S. Cl. 552/544; 514/182

(57) ABSTRACT

This invention relates to the field of estrogen receptors and particularly though not exclusively on the effect of estrogen receptors and ligands for estrogen receptors on the prevention or treatment of obesity. The invention also relates to the effect of estrogen receptors and their ligands on lipoprotein levels in mammals.

This application claims the benefit of U.S. Provisional Application Nos. 60/275,023; 60/274,996; 60/275,047; and 60/274,995, all filed Mar. 12, 2001.

### BACKGROUND OF THE INVENTION

1. Field of the Invention

This invention relates to the field of estrogen receptors and particularly, though not exclusively, to the effect of estrogen receptors and ligands for estrogen receptors, particularly those ligands which are agonists, and on the use of those ligands for prevention or treatment of obesity. The invention also relates to the effect of estrogen receptors and their ligands on rut lipoprotein levels in mammals.

2. Description of the Related Art

The cloning of the novel estrogen receptor, ERβ, suggested that there may exist alternative mechanisms of action for estrogen (Kuiper, G. G., et al (1996) Proc. Natl. Acad. Sci. USA 93, 5925-5930). For example, ERβ is expressed in growth plate chondrocytes and osteoblasts, indicating a possible role for ERβ in the regulation of longitudinal bone growth and/or adult bone metabolism (Onoe, Y., et al (1997) Endocrinology 138, 4509-4512; Arts, J., Kuiper, G. G., et al (1997) Endocrinology 138, 5067-5070; Vidal, O., et al (1999) J Bone Miner Res In press; Nilsson, L. O., et al (1999) J Clin Endocrinol Metab 84, 370-373; Windahl own unpublished results). We have recently generated mice devoid of functional ERβ protein and reported that ERβ is essential for normal ovulation efficiency, but is not essential for female or male sexual development, fertility, or lactation (Krege, J. H., et al (1998) Proc Natl Acad Sci USA 95, 15677-15682).

The molecular mechanisms of action for ERα compared to ERβ have recently been investigated. ERα and ERβ have almost identical DNA-binding domains and studies in vitro have demonstrated that the two receptors have similar affinities for estrogenic compounds (Kuiper, G. G. et al (1996) Proc Natl Acad Sci USA 93, 5925-5930; Kuiper, G. G., et al (1997) Endocrinology 138, 863-870; Tremblay, G. B., et al (1997) Mol Endocrinol 11, 353-365). The amino-acid sequence of ERβ differs from ERα. in the N- and C-terminal trans-activating regions. Therefore the transcriptional activation mediated by ERβ may be distinct from that of ERα (Paech, K., et al (1997) Science 277, 1508-1510). Considering the great similarities in ligand- and DNA-binding specificity, it has been speculated that a differential tissue distribution of estrogen receptors may be important for mediating tissue specific responses to estrogens (Kuiper, G. G., and Gustafsson, J. A. (1997) FEBS Lett 410, 87-90). Thus, the unique transactivating domains of the two receptor subtypes, in combination with differential tissue-distribution, or differential cell-type distribution within a tissue, could be important factors to determine the estrogen response in target tissues.

It is well known that estrogen exerts atheroprotective effects in women. The incidence of atherosclerotic disease is low in premenopausal women, rises in postmenopausal women, and is reduced in postmenopausal women who receive estrogen therapy Mendelsohn M E, Karas R H, N Engl J Med (1999) 340, 1801-1811; Stampfer M J et al (1991) N Engl J Med 325, 756-762; Grady D et al (1992) Ann Intern Med 117,1016-1027; Barrett-Connor E (1997) Circulation 95, 252-264). The protective effect of estrogen depends both on estrogen induced alterations in serum lipids and on direct actions of estrogen on blood vessels (Mendelsohn M E, Karas R H, (1999) supra). The possible protective effects of estrogen in males are less well documented. However, recent clinical findings in males with either aromatase deficiency (estrogen deficient) or estrogen resistance (estrogen receptor mutation) have indicated that estrogen exerts important effects on carbohydrate and lipid metabolism in males as well (Smith E P et al (1994) N Engl J Med 331, 1056-1061; Morishima A et al (1995) J Clin Endocrinol Metab 80, 3689-3698; Grumbach M M et al (1999) J Clin Endocrinol Metab 84, 4677-4694). The clinical features of !L these patients include glucose intolerance, hyperinsulinemia and lipid abnormalities (MacGillivray M H et al (1998) Horm Res 49 Suppl 1, 2-8). Furthermore, estrogen resistance in a male subject was associated with premature coronary atherosclerosis (Grumbach M M et al (1999) supra).

Orchidectomy (orx) results both in a decreased activation of the androgen receptor and decreased estrogen levels, leading to decreased activation of estrogen receptors. We have previously demonstrated that orx of male mice results in a decreased weight gain during sexual maturation (Sandstedt J et al (1994) Endocrinology 135, 2574-2580). Similarly, orx of rats also results in a decreased body weight (Vanderschueren D et al (1996) Caldif Tissue Int 59 179-183; Vanderschueren D et al (1997) Endocrinology 138 2301-2307; Zhang X Z et al (1999) Bone Miner Res 14 802-809). However, the decreased body weight in orchidectomized mice and rats was accompanied by a decreased size of the skeleton, indicating that it is a growth related effect rather than an effect related to the fact that the animals became leaner. The effect of estrogen on fat content, carbohydrate metabolism and lipid metabolism in male mice is largely unknown. However, it was recently reported that aromatase deficient (ArKO) male mice, with decreased serum levels of estrogen, had a 50% increase of the gonadal fat pads (Fisher CR et al (1998) Proc Natl Acad Sci USA 95 6965-6970). No information about carbohydrate and lipid metabolism in these mice was given in that publication.

Possible effects of estrogen on fat mass may, for instance, include direct effects on the fat tissue and indirect central effects on food intake, food efficiency and activity. Furthermore, it is known that estrogen exerts liver specific effects on lipid and carbohydrate metabolism. The two estrogen receptor subtypes, ERα and ERβ, bind estrogen with similar affinity but are believed to differ in their transactivating properties. The relative importance of ERα and ERβ in adipose tissue is not known. Some previous studies have reported ERα protein (Mizutani T et al (1994) J Clin Endocrinol Metab 78, 950-954; Pedersen S B et al (1996) Eur J Clin Invest 26, 1051-1056) as well as specific estrogen binding and ERβ mRNA to be present in human subcutaneous adipose tissue (Pedersen S B et al (1996) supra). However, others have failed to detect estrogen receptors in human adipose tissue (Bronnegard M et al (1994) J Steroid Biochem Mol Biol 51, 275-281; Rebuffe-Scrive M et al (1990) J Clin Endocrinol Metab 71, 1215-1219). More recently, ERβ mRNA has been detected in human subcutaneous adipose tissue, suggesting that direct effects of estrogen may involve both receptor subtypes (Crandall D L et al (1998) Biochem Biophys Res Commun 248, 523-526).

Mice lacking a functional ERα gene, ERα Knockout mice (ERKO), have been generated (Couse, J. F. et al (1995) Mol. Endocrinol. 9, 1441-1454) and more recently ERG Knockout mice (BERKO) have also been described (Krege, J. H. et al (1998) Proc. Natl. Acad. Sci. USA 95, 15677-15682). We have also generated Double-ER-Knockout mice (DERKO) i.e. mice having no estrogen receptors.

### SUMMARY OF THE INVENTION

The aim of the present study was to investigate the function of the estrogen receptors and in particular their effects on body fat and serum levels of leptins in mammals. These parameters were studied in ERα knockout (ERKO), ERβ knockout (BERKO) and ERα/β double knockout (DERKO) mice before during and after sexual maturation.

Surprisingly, it was found that neither the total body fat nor serum leptin levels were altered in any group before or during sexual maturation. However, after sexual maturation, ERKO and DERKO but not BERKO demonstrated a markedly increased amount of total body fat as well as increased serum levels of leptin. Serum levels of corticosterone were decreased whereas serum cholesterol was increased in adult mice with ERα inactivated. Interestingly, a qualitative change in the lipoprotein profile, including smaller and denser LDL particles, was also observed in ERKO and DERKO mice. In conclusion, ERα but not ERβ inactivated male mice develop obesity after sexual maturation. This obesity is associated with a disturbed lipoprotein profile.

It is well known that ovariectomy (ovx) in the rat results in weight gain, which, at least in part, is due to an increase in food intake (Bennett P A et al (1998) Neuroendocrinology 67, 29-36; Richter C et al (1954) Endocrinology 54, 323-337). Conversely, estrogen is well known to suppress food intake and reduce body weight in female rats (Couse, J. F. & Kovach K. S. (1999) Science, 286, 2328; Mook D G et al (1972) J Comp Physiol Psychol 81, 198-211). A weight reducing effect of estrogen in female rodents is supported by the fact that female ArKO mice, with undetectable levels of estrogen, develop increased weight of the mammary- and the gonadal-fat pads after sexual maturation (Fisher C R et al (1998 supra). It is unknown whether or not estrogen reduces body weight in male rodents. We have in the present study demonstrated that adult male mice, devoid of all known estrogen receptors, develop obesity, indicating that estrogen reduces body weight in male rodents as well. A physiological fat reducing effect of estrogen in males is supported by a recent observation that the weight of the gonadal fat pads is increased in male ArKO mice. Furthermore, the estrogen receptor specificity for this obese phenotype in DERKO and ArKO mice was investigated. In the present study, ERα but not ERβ inactivated mice developed a similar obese phenotype as did the DERKO mice, demonstrating that ERα inactivation is responsible for the obese phenotype in DERKO mice. In contrast, a non significant tendency of reduced weight of the retroperitoneal fat pads was found in male BERKO mice. We are currently feeding BERKO and wild type mice with high fat diet in order to investigate whether or not BERKO mice actually are less obese than wild type mice. The mechanism behind the adult obesity in ERα-inactivated mice is unknown and may include both peripheral and central effects.

Serum levels of IGF-I are decreased in ERKO and DERKO mice and clinical studies have demonstrated that male obesity is associated with low serum levels of IGF-I (Vidal 0 et al (2000) Proc Natl Acad Sci USA in press; Bennett P A et al (1998) supra; Richter C et al (1954) supra; Mook D G et al (1972) supra; Marin P et al (1993) Int J Obes Relat Metab Disord 17, 83-89). Thus, one possible mechanistic explanation for the increased fat mass in ERKO and DERKO mice might be a reduction of serum IGF-I levels, resulting in obesity.

Estrogen therapy reduces the risk of developing cardiovascular disease (Psaty B M et al (1993) Arch Intern Med 153 1421-1427; The writing group for the PEPI t 1995) JAMA 273 199-208; Grodstein F et al (1996) N Engl J Med 335 453-461; Henriksson P et al (1989) Eur J Clin Invest 19 395-403; Wagner J D et al (1991) J Clin Invest 88 1995-2002; Haabo J et al (1994) Arterioscler Thromb 14 243-247; Herrington D M et al (1994) Am J Cardiol 73 951-952; Zhu X D et al (1997) Am J Obstet Gynecol 177 196-209). The ability of estrogen to lower plasma levels of total cholesterol and to reduce plasma level of LDL-particles is of importance for the cardioprotective effect of estrogen since elevated levels of cholesterol are strongly associated with cardiovascular disease (Gordon T et al (1981) Arch Intern Med 141, 1128-1131). The higher exposure to estrogens in females than males has been proposed as being the protective factor explaining the lower risk for cardiovascular disease that women have compared with men (Kannel W B et al (1976) Ann Intern Med 85, 447-452; Bush T L et al (1990) Ann NY Acad Sci 592, 263-71). The protective effects of estrogen in preventing atherosclerosis have also been Hi described in animal models (Henriksson P et al (1989) supra; Kushwaha R S et al (1981) Metabolism 30, 359-366). At least some of the effects of estrogens on cholesterol metabolism have been shown to be dependent on ERs (Parini, P et al (1997) Arterioscler Thromb Vasc Biol 17, 1800-1805; Scrivastava R A et al (1997) JBiol Chem 272, 33360-33366). However, the physiological role exerted by ERs in the regulation of cholesterol and lipoprotein metabolism is still unclear.

Clinical case reports have described that estrogen resistance results in metabolic effects including disturbed lipid profile (Smith E P et al (1994) supra). In the present study, the levels of total cholesterol were increased in ERα but not in ERβ inactivated male mice. Furthermore, the disruption of the ERα gene, alone or in association with the disruption of the ERβ gene, resulted in an atherogenic lipoprotein profile characterized by an increase in the smaller and denser LDL particles. This atherogenic lipoprotein profile was not present in male BERKO mice, denoting a clear phenotype associated with the ERα and suggesting a physiological role for ERα in the regulation of lipoprotein metabolism in male mice.

The mechanism behind the altered lipoprotein profile in male ERα-inactivated mice cannot be decided from the present study, but may for instance include alterations in serum levels of apolipoprotein E, hepatic lipase activity and LDL-receptor expression. It has previously been described that wild type mice, but not ERKO mice, display an estrogen induced increase in serum levels of apolipoprotein E. In contrast, the basal apolipoprotein E levels were not significantly decreased in ERKO mice compared with wild type mice (Scrivastava R A et al (1997) J Biol Chem 272, 33360-33366). Estrogen administration to mice does not affect the activity of hepatic lipase (Scrivastava R A et al (1997) Mol Cell Biochem 173, 161-168). However, this finding does not rule out the possibility that ER-inactivation may regulate hepatic lipase activity. Difference in LDL-receptor expression should also be considered. High dose estrogen treatment increases LDL-receptor expression in rats (Kovanen PT et al (1979) J Biol Chem 254, 11367-11373; Chao Y S et al (1979) J Biol Chem 254, 11360-11366), rabbits (Henriksson P et al (1989) supra; Ma PT et al (1986) Proc Natl Acad Sci USA 83, 792-796) and human (Angelin B et al (1992) Gastroenterology 103, 1657-1663). In contrast, treatment of rats with antiestrogens does not reduce hepatic LDL-receptor expression (Parini P et al (1997) Arterioscler Thromb Vasc Biol 17, 1800-1805) and LDL-receptors are not upregulated by estrogen in mice (Scrivastava R A et al (1997) supra; Scrivastava R A et al (1994) Eur J Biochem 222, 507-514), suggesting that LDL-receptor expression is not dependent on ERs in mice.

ERKO and DERKO but not BERKO mice had increased levels of cholesterol in the HDL-fraction, supporting previous reports that administration of estrogen decreases HDL-cholesterol levels in mice (Tang J J et al (1991) 32, 1571-1585). In contrast, estrogen increases HDL-cholesterol in humans. Furthermore, the insulin×glucose as well as the insulin×free fatty acid products were increased in the ERα inactivated mice, indicating that these mice are insulin resistant. Clinical studies have demonstrated that men with defective estrogen synthesis or action also have a propensity for both insulin resistance and dyslipidemia (Smith EP et al (1994) supra; Morishima A et al (1995) supra; Grumbach M M et al (1999) supra). These men, as well as DERKO and ArKO mice, have increased serum levels of testosterone (Fisher C R et al (1998) supra; Vortkamp A et al (1996) Science 273, 613-622). The role of a high concentration of testosterone (or its action in the absence of estrogen) is uncertain. Estrogen therapy reverses the lipid abnormalities seen in men with estrogen deficiency (Grumbach M M et al (1999) J Clin Endocrinol Metab 84, 4677-4694). Correction of the lipid abnormalities could either be a direct effect of estrogen or an indirect effect via normalization of the high serum androgen concentration.

Selective estrogen receptor modulators (SERMs) have been shown to maintain estrogen's positive bone and cardiovascular effects while minimizing several of the side-effects of estrogen (Delmas P D et al 1997) N Engl J Med 337, 1641-1647). It has been well documented that SERMs decrease total serum cholesterol in ovx female rats (Bryant H et al (1996) Jounral of Bone and Mineral Metabolism 14, 1-9; Black L J et al (1 994) J Clin Invest 93, 63-69; Ke H Z et al (1997) Bone 20, 31-39) and total serum cholesterol and low density lipoprotein in postmenopausal women (Delmas P D et al (1997) supra; Cosman F et al (1999) Endocr Rev 20, 418-434). Furthermore, oral estrogen treatment improves serum lipid levels in elderly men (Giri S et al (1998) Atherosclerosis 137, 359-366). A recent study demonstrated that the SERM lasofoxifene decreased total serum cholesterol in orx male rats, indicating that lasofoxifene acts as an estrogen agonist for serum lipoproteins in male rats, similar to that seen in ovx female rats (Ke H Z et al (2000) Endocrinology 141, 1338-1344). Lasofoxifene treated orx male rats demonstrated decreased food intake and body weight, which may result in the decreased total serum levels of cholesterol. The result that lasofoxifene decreases body weight and serum levels of cholesterol in male mice is consistent with the present study in which male ER-inactivated mice develop obesity and increased serum levels of cholesterol.

It has recently been demonstrated that mice devoid of all known ERs are viable (Vidal O et al (2000) supra; Couse J F et al (1999) Science 286, 2328-2331). However, loss of both receptors leads to an ovarian phenotype that is distinct from that of the individual ER mutants indicating that both receptors are required for the maintenance of germ and somatic cells in the postnatal ovary (Couse J F et al (1999) supra). Furthermore, the skeletal growth is inhibited in male DERKO mice, associated with decreased serum levels of IGF-I (Vidal O et al (2000) supra). Dissection of the estrogen receptor specificity clearly demonstrated that ERα but not ERβ was responsible for the inhibited growth seen in DERKO mice (Vidal O et al (2000) supra). The present data represents the first information about the metabolic phenotype of DERKO mice. Similar to the growth related effects, the metabolic effects, including the reduction of fat described in the present study, seem to be mediated via ERα and not ERβ. Therefore, one may speculate that ERα specific agonists could be useful in the treatment of some males with obesity and/or disturbed lipoprotein profile. In conclusion, ERα inactivated male mice develop obesity after sexual maturation. This obesity is associated with a disturbed lipoprotein profile.

### BRIEF DESCRIPTION OF THE DRAWINGS

The invention will now be described, by way of example only, with reference to the accompanying drawings FIGS. 1 to 6 in which:

FIG. 1 shows total body fat levels in wild type (WT), ERKO, BERKO and DERKO mice before sexual maturation, during sexual maturation and after sexual maturation;

FIG. 2 shows serum leptin levels in wild type (WT), ERKO, BERKO and DERKO mice before sexual maturation, during sexual maturation and after sexual maturation;

FIG. 3 shows fat content in sexually mature male wild type (WT) ERKO, BERKO and DERKO mice;

FIG. 4 shows dissected retroperitoneal and gonadal fat levels in sexually mature male wild type (WT), ERKO, BERKO and DERKO mice;

FIG. 5 shows serum lipoprotein levels in sexually mature male wild type (WT) mature male wild type (WT) ERKO, BERKO, and DERKO mice; and

FIG. 6 shows the effect of estrogen on fat levels in wild type (WT) ERKO, BERKO and DERKO mice.

### DETAILED DESCRIPTION OF THE INVENTION

According to one aspect of the invention, there is provided the use of an ERα selective compound in the preparation of a medicament for the treatment or prevention of obesity in a mammalian subject. The invention also provides a method of treating or preventing obesity in a mammalian subject comprising supplying an ERα selective compound to the subject. Preferably, the ERα selective compound is an ERα agonist. The mammalian subject may preferably be adult although the treatment of sexually maturing mammals is contemplated. The mammalian subject may be human, but the treatment of other species, especially domesticated species, is also contemplated. Gonadal fat levels may be reduced as a percentage of body weight to about 1.25% or below.

The invention also provides a pharmaceutical composition for the treatment or prevention of obesity, the composition comprising an ERα selective compound, preferably an ERα agonist. Pharmaceutical compositions of this invention comprise any of the compounds of the present invention, and pharmaceutically acceptable salts thereof, with any pharmaceutically acceptable carrier, adjuvant or vehicle. Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamnine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions of this invention may be administered orally, parenterally, by inhalation spray, topically, rectally, nasally, buccally, vaginally or via an implanted reservoir. We prefer oral administration or administration by injection. The pharmaceutical compositions of this invention may contain any conventional non-toxic pharmaceutically-acceptable carriers, adjuvants or vehicles. The term parenteral as used herein includes subcutaneous, intracutaneous, intravenous, intramuscular, intra-articular, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant such as Ph. Helv or a similar alcohol.

The pharmaceutical compositions of this invention may be orally administered in any at orally acceptable dosage form including, but not limited to, capsules, tablets, and aqueous suspensions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are administered orally, the active ingredient is M combined with emulsifying and suspending agents. If desired, certain sweetening and/or flavoring and/or coloring agents may be added.

The pharmaceutical compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this invention is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches are also included in this invention.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

In a pharmaceutical composition of the invention the ER(X selective compound is preferably an ERα agonist.

The invention also provides a method of screening compounds for efficiacy in the treatment or prevention of obesity, the method including determining the ER binding properties of the components. The compounds are preferably selected on the basis of being ERα selective compounds. Most preferably compounds are selected which are ERα agonists.

According to another aspect of the invention there is provided an ER(X selective compound in the preparation of a medicament for the reduction or lowering of serum lipoprotein levels in a mammalian subject. The ERα selective compound is preferably an ERα agonist. The ERα agonist is preferably ERα selective. The subject is preferably adult, most preferably human.

The invention also provides pharmaceutical composition for the reduction of serum lipoprotein levels, the composition comprising an ERα selective compound. The ERα selective compound is preferably an ERα agonist. The invention also provides a method of screening compounds for efficiacy in the reduction of serum lipoprotein levels, the method including determining the ER binding properties of the compounds. Compounds are preferably selected on the basis of being ERα agonists. Preferably the agonists are selective for ERα.

Definitions

“ER Agonism”: An ER agonist is a compound that displays ≧50% of the activity of the natural estrogen 17β-estradiol (E2) or the synthetic estrogen moxestrol, activity defined as e.g the increased expression of a gene product that is transcriptionally controlled by an estrogen-response-element (ERE)-promoter-gene construct (ERE-reporter vector) in the presence of an ER.

“ER antagonism”: An ER antagonist is a compound that displays ≦5% or no agonist activity compared to the activity displayed by the natural estrogen 17β-estradiol (E2) or the synthetic estrogen moxestrol, or a compound that decrease the activity of E2 or the synthetic estrogen moxestrol down to ≦5% of the activity displayed by E3 or the synthetic estrogen moxestrol alone, activity defined as e.g the increased expression of a gene product that is transcriptionally controlled by an estrogen-response-element (ERE)-promoter-gene construct (ERE-reporter vector) in the presence of an ER.

“Compound with mixed agonist/antagonist activity”. (SERM: Selective Estrogen Receptor Modulator): An ER-binding compound that displays ≦50% but ≧5% of the activity of the natural estrogen 17β-estradiol (E2) or the synthetic estrogen moxestrol, activity defined as e.g the increased expression of a gene product that is transcriptionally controlled by an estrogen-response-element (ERE)-promoter-gene construct (ERE-reporter vector) in the presence of an

“ERa selective compound”: An ERα selective compound is a compound that displays ≧10-fold higher binding affinity for ERα than for ERβ as determined by a standard receptor-ligand competition binding assay, and/or that displays ≧10-fold higher potency via ERα than via ERβ in the transcriptional regulation of an estrogen sensitive gene in the presence or absence of the natural estrogen 17β-estradiol (E2) or the synthetic estrogen moxestrol. Estrogen sensitive gene defined by an estrogen-response-element (ERE)-promoter-gene construct (ERE-receptor vector).

“ERβ selective compound”: An ERβ selective compound is a compound that displays ≧10-fold higher binding affinity for ERβ than for ERα as determined by a standard receptor-ligand competition binding assay, and/or that displays ≧10-fold higher potency via ERβ than via ERα in the transcriptional regulation of an estrogen sensitive gene in the presence or absence of the natural estrogen 17β-estradiol (E2) or the synthetic estrogen moxestrol. Estrogen sensitive gene defined by an estrogen-response-element (ERE)-promoter-gene construct (ERE-reporter vector).

“ERα selective agonist”: An ERα selective agonist is a compound that displays ≧50% of the activity of the natural estrogen 17β-estradiol (E2) or the synthetic estrogen moxestrol, mediated by ERα, but ≦50% of the activity of the natural estrogen 17β-estradiol (E2) or the synthetic estrogen moxestrol, mediated by ERA. Activity defined as e.g the increased expression of a gene product that is transcriptionally controlled by an estrogen-element (ERE)-promoter-gene construct (ERE-reporter vector) in the presence of ERα or ERβ.

“ERβ selective agonist”: An ERβ selective agonist is a compound that displays ≧50% of the activity of the natural estrogen 17β-estradiol (E2) or the synthetic estrogen moxestrol, mediated by ERβ, but ≦50% of the activity of the natural estrogen 17β-estradiol (E2) or the synthetic estrogen moxestrol, mediated by ERα. Activity defined as e.g the increased expression of a gene product that is transcriptionally controlled by an estrogen-response-element (ERE)-promoter-gene construct (ERE-reporter vector) in the presence of ERβ or ERα.

“ERα selective compound with mixed agonist/antagonist activity (SERM: Selective Estrogen Receptor Modulator)”: An ER-binding compound that displays ≦50% but ≦5% of the activity of the natural estrogen 17β-estradiol (E2) or the synthetic estrogen moxestrol, mediated by ERα:, but ≧50% or ≦5% of the activity of the natural estrogen 17β-estradial (E2) or the synthetic estrogen moxestrol, mediated by ERβ. Activity defined as e.g the increased expression of a gene product that is transcriptionally controlled by an estrogen-response-element (ERE)-promoter-gene construct (ERE-reporter vector) in the presence of ERα or ERβ.

“ERβ selective compound with mixed agonist/antagonist activity (SERM Selective Estrogen Receptor Modulator)”: An ER-binding compound that displays ≦50% but ≧5% of the activity of the natural estrogen 17β-estradiol (E2) or the synthetic estrogen moxestrol, mediated by ERβ, but ≧50% or ≦5% of the activity of the natural estrogen 17β-estradiol (E2) or the synthetic estrogen moxestrol, mediated by ERα. Activity defined as e.g the increased expression of a gene product that is transcriptionally controlled by an estrogen-response-element (ERE)-promoter-gene construct (ERE-reporter vector) in the presence of ERβ or ERα.

“ERα selective antagonist”: An ER-binding compound that displays ≦5% or no agonist activity compared to the activity displayed by the natural estrogen 17β-estradiol (E2) or the synthetic estrogen moxestrol, mediated by ERα, but that displays ≧5% of the activity of the natural estrogen 17β-estradiol (E2) or the synthetic estrogen moxestrol, mediated by ERG. Activity defined as e.g, the increased expression of a gene product that is transcriptionally controlled by an estrogen-response-element (ERE)-promoter-gene construct (ERE-reporter vector) in the presence of ERα or ERβ.

“ERβ selective antagonist”: An ER-binding compound that displays ≦5% or no agonist activity compared to the activity displayed by the natural estrogen 17β-estradiol (E2) or the synthetic estrogen moxestrol, mediated by ERβ, but that displays ≧5% of the activity of the natural estrogen 17β-estradiol (E2) or the synthetic estrogen moxestrol, mediated by ERα. Activity defined as e.g the increased expression of a gene product that is transcriptionally controlled by an estrogen-response-element (ERE)-promoter-gene construct (ERE-reporter vector) in the presence of ERβ or ERα.

### EXAMPLES

The invention is further described by the following Examples, but is not intended to be limited by the Examples. All parts and percentages are by weight and all temperatures are in degrees Celsius unless explicitly stated otherwise.

1. Methods

a) Animals

Male double heterozygous (ERα^{+/-}β^{+/-}) mice were mated with female double heterozygous (ERα^{+/-}β^{+/-}) mice, resulting in WT, ERKO, BERKO and DERKO offspring. All mice were of mixed C57BL/6J/129 backgrounds. Genotyping of tail DNA was performed at 3 weeks of age. The ER α-gene was analyzed with the following primer pairs: Primers AACTCGCCGGCTGCCACTTACCAT and CATCAGCGGGCTAGGCGACACG for the WT gene correspond to flanking regions in the targeted exon no. 2. They produce a fragment of approximately 320 bp. Primers TGTGGCCGGCTGGGTGTG and GGCGCTGGGCTCGTTCTC for the KO gene correspond to part of the NEO-cassette and the flanking exon no. 2. They produce a 700 bp fragment. Genotyping of the ERβ-gene has been previously described (Windahl S. H. et al (1999) J. Clin Invest 104: 895-901). Animals were maintained in polycarbonate plastic cages (Scanbur A S, Køge, Denmark) containing wood chips. Animals had free access to fresh water and food pellets (B&K Universal AB, Sollentuna, Sweden) consisting of cereal products (76.9% barley, wheat feed, wheat and maize germ), vegetable proteins (14.0% hipro soya) and vegetable oil (0.8% soya oil).

b) Dual X-Ray Absorptiometry (DXA)

We have previously developed a combined Dual X-Ray Absorptiometry (DXA) Image analysis procedure for the in vivo prediction of fat content in mice (Sjogren et al manuscript). The DXA measurements were done with the Norland pDEXA Sabre (Fort Atkinson, Wis.) and the Sabre Research software (3.9.2). Three mice were analysed in each scan. A mouse, which was sacrificed at the beginning of the experiment, was included in all the scans as an internal standard in order to avoid inter-scan variations. The software % fat procedure was used with a setting so that areas with more than 50% fat was made white on the image. The accuracy of this setting was checked daily with a standard consisting of a gradient with 0-100% fat. The image was then printed, scanned and imported to the software Scion Image (Scion Corporation, Frederick, Md.). The imported image was then threshold to a setting of 50 arbitrary units, making lean mass and bone black while the fat area appeared as white holes in the mice. Therafter, the “analyse particle” procedure was performed first with white areas in mice included (=A1=total mouse area) and then without the white area included (=A2=lean area+bone area). The % fat area was then calculated as ((A1−A2)/A1)×100. The inter-assay CV for the measurements of % fat area was less than 3%.

c) Serum Levels of Leptin, Insulin, Corticosterone, Cholesterol, Triglycerides, Glucoso and Free Fatty Acids

Serum leptin levels were measured by a radio immuno assay (Chrystal Chem Inc, IL, USA) with an intra-assay and interassay coefficient of variations (CVs) of 5.4 and 6.9%, respectively. Serum insulin levels were measured by a radio immuno assay (Chrystal Chem Inc, IL, USA) with an intra-assay and interassay coefficient of variations (CVs) of 3.5 and 6.3%, respectively. Serum corticosterone levels were measured by a radio immuno assay (ImmunoChem ICN Biomedicals, Inc CA USA) with an intra-assay and interassay coefficient of variations (CVs) of 6.5 and 4.4%, respectively. Serum total cholesterol, triglycerides and glucose were assayed using the respective commercially available assay kit from Boebringer Mannheim (Mannheim, Germany). Free fatty acids were measured by an enzymatic colorimetric method (ACS-ACOD; Wako Chemicals Inc, VA, USA) with an intra-assay coefficient of variations (CV) of less than 3%.

d) Lipoprotein Cholesterol Determination

Size fractionation of lipoproteins by miniaturized on-line FPLC was performed using a micro-FPLC column (30×0.32 cm Superose 6B) coupled to a system for on-line detection of cholesterol. In brief, 10 μl of serum from each animal was injected and the cholesterol content in the lipoproteins was determined on-line using a cholesterol assay kit (Boehringer Mannheim, Mannheim, Germany), which was continuously mixed with the separated lipoproteins. Absorbance was measured at 500 nm and the signals collected using EZ CROM software (Scientific Software, San Ramon, Calif.).

e) Effects of Estrogen Exposure

Male double heterozygous (ERα^{+/-}β^{+/-}) mice were mated with female double heterozygous (ERα^{+/-}β^{+/-}) mice, resulting in ERα^{+/+}β^{+/+}=wildtype (WT); ERα^{-/-}β^{+/+}=ERKO, ERα^{+/+}β^{-/-}=BERKO and ERα^{-/-}β^{-/-}=DERKO offsprings (Vidal O et al (2000) Proc Natl Sci USA, 97, 5474). The diet, housing and genetic background was as previously described in Vidal O et al (2000) supra. In the estrogen exposure experiments all mice were ovariectomized. Ovaries were removed after a flank incision and the incisions were closed with metallic clips. Mice were left to recover for four days after ovariectomy before start of experiments. After recovery mice were injected s.c with 2.3 μg/mouse/day of 17β-estradiol benzoate (Sigma, St Louis, Mo., USA) for 5 days/week during three weeks time. Control mice received injections of vehicle oil (olive oil, Apoteksbolaget, Göteborg, Sweden).

2) Results

a) Measurement of Body Fat Levels

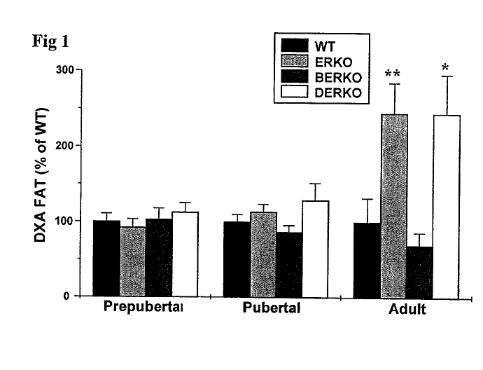

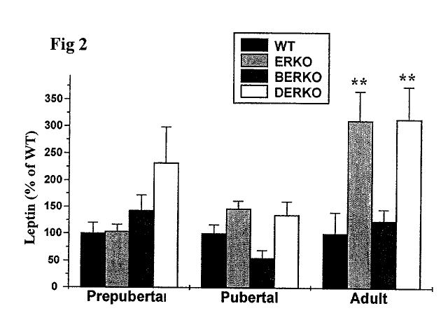

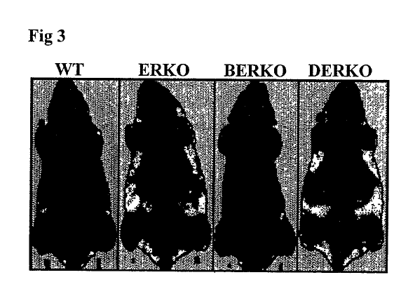

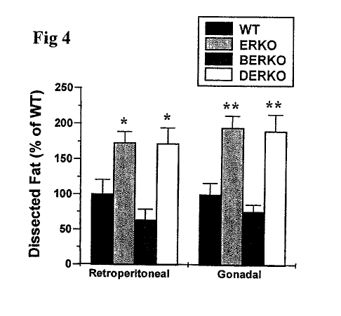

We have previously demonstrated that male ERKO and DERKO mice develop a retarded longitudinal bone growth concomitantly with a reduced body weight gain during sexual maturation (Vidal O et al (2000) Proc Nati Acad Sci USA in press). However, two months after sexual maturation, no significant effect on body weight was seen in ERKO and DERKO (4 months of age; WT 33.0±1.1 g, ERKO 31.6±0.9 g, BERKO 31.1±0.6 g, DERKO 33.0±1.6 g). Thus, the 4 months old ERKO and DERKO mice had decreased size of the skeleton while their body weight was unchanged, indicating that they had become obese. Therefore, the serum levels of leptin and total body fat content, as measured with DXA, were followed before, during and after sexual maturation in male wt, ERKO, BERKO and DERKO mice. Neither the total body fat nor serum leptin levels were altered in any group before (1 months of age) or during (2 months of age) sexual maturation (FIGS. 1-2). Specifically FIG. 1 shows total body fat, as measured using dual energy X-ray absorptiometry, in wild type (WT), ERKO, BERKO and DERKO mice before sexual maturation (Prepubertal, 1 month of age), during sexual maturation (Pubertal, 2 months of age) and after sexual matruation (Adult, 4 months of age; n=5-9). Values are given as means±SEM. Data were first analysed by a one-way analysis of variance followed by Student-Neuman-Keul's multiple range test. In FIG. 1 *p<0.05 versus WT, **p<0.01 versus WT. FIG. 2 shows serum leptin levels in wild type (WT), ERKO, BERKO and DERKO mice before sexual maturation (Prepubertal, 1 month of age), during sexual maturation (Pubertal, 2 months of age) and after sexual maturation (Adult, 4 months of age; n=5.9). Values are given as means±SEM. Data were first analysed by a one-way analysis of variance followed by Student-Neuman-Keul's multiple range test *p<0.05 versus WT. In FIG. 2 **p<0.01 versus WT. However, after sexual maturation (4 months of age), ERKO and DERKO but not BERKO demonstrated a markedly increased amount of total body fat as well as increased serum levels of leptin (FIGS. 1-3). FIG. 3 shows DXA/Image analysis of fat content in mice. Four months old male wild type (WT), ERKO, BERKO and DERKO mice were scanned in a DXA, followed by Image analysis as described above. Areas with more than 50% fat are shown as white areas while areas with leam mass and bone are shown as black areas. The increased amount of fat in adult (four month old) ERKO and DERKO mice was also reflected in a pronounced increase in the weight of dissected retroperitoneal and gonadal fat (FIG. 4). In FIG. 4 values are given as means±SEM. Data were first analysed by a one-way analysis of variance followed by Student-Newman-Keul's multiple range test. *p<0.05 versus WT, **p<0.01 versus WT. In contrast a non significant tendency of reduced weight of the retroperitoneal fat pads was found in ERβ inactivated male mice (−37%, p=0.02, FIG. 4).

b) Measure of Metabolic Serum Parameters

No significant effect in any group was seen on serum levels of insulin, free fatty acids or triglycerides (Table 1).

TABLE 1
Metabolic Serum Parameters

| | | | | | 2-way | |
|---|---|---|---|---|---|---|
| | WT | ERKO | BERKO | DERKO | ANOVA | |
| | (n = 6) | (n = 9) | (n = 6) | (n = 5) | ERα—/— | |
| Corticosterone (ng/ml) | 135 ± 34 | 67 ± 8 | 139 ± 15 | 96 ± 35 | P < 0.05 | NS |
| Insulin (pg/ml) | 389 ± 42 | 352 ± 33 | 308 ± 12 | 454 ± 40 | NS | NS |
| Glucose (mM) | 27.9 ± 1.0 | 30.3 ± 1.0 | 23.5 ± 0.9* | 31.6 ± 2.0 | P < 0.01 | NS |
| Free Fatty Acids | 1.09 ± 0.0 | 1.32 ± 0.08 | 1.05 ± 0.12 | 1.15 ± 0.08 | NS | NS |
| (mEq/l) | 8 | | | | | |
| Insulin × Glucose | 10.9 ± 1.4 | 11.2 ± 0.9 | 7.2 ± 0.3* | 15.2 ± 1.4* | P < 0.01 | NS |
| FFA × Insulin | 420 ± 44 | 473 ± 61 | 323 ± 39 | 505 ± 32 | P < 0.05 | NS |
| Cholesterol (nM) | 3.22 ± 0.1 | 3.52 ± 0.23 | 2.85 ± 0.22 | 3.55 ± 0.20 | P < 0.05 | NS |
| | 6 | | | | | |
| Triglycerides (nM) | 1.49 ± 0.1 | 2.18 ± 0.23 | 1.70 ± 0.35 | 1.83 ± 0.13 | NS | NS |
| | 7 | | | | | |

Values are given as means±SEM. Data were first analysed by a one-way analysis of variance followed by Student-Neuman-Keul's multiple range test *p<0.05 versus WT. Furthermore, a 2-way analysis of variance followed by Student-Neuman-Keul's multiple range test was performed, in which ERα^{-/-} and ERβ^{-/-} was regarded as separate treatments. The p-value versus respective +/+ allele is indicated. NS=non significant.

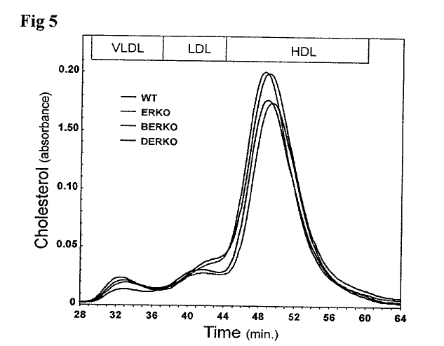

However, the insulin x glucose as well as the insulin×free fatty acid products were increased in the ERα. inactivated mice (2 way-ANOVA; Table 1), indicating that these mice are insulin resistant. Furthermore, the serum levels of corticosterone were decreased while serum levels of glucose and cholesterol were increased in mice with ERα inactivated (2 way-ANOVA; Table 1). In order to study the effects on serum cholesterol in more detail, lipoproteins were separated by micro-FPLC and their cholesterol content was determined on-line in 4 months old male wild type (WT), ERKO, BERKO and derko MICE (N=5−9). After separation of 10 μl serum from each animal, cholesterol content in lipoproteins was determined on-line and the absorbance measured at 500 nm. Mean profiles are shown. (FIG. 5). An increased high density lipoprotein (HDL) peak was found in adult male ERKO and DERKO but not in BERKO mice. Interestingly, the ERKO and DERKO mice had a qualitative alteration in the low density lipoprotein (LDL) peak, resulting in an increase of cholesterol in the smaller LDL particles.

C) Measurement of Gonadal Fat

Ovariectomized (ovx) mice, lacking one or both of the two known ERs, were given estrogen and the effects on gonadal fat was studied. The effects of estrogen in mice with both ERα and ERβ inactivated (DERKO) were compared with the effects of estrogen in wild type (WT) mice. Estrogen treatment of ovx WT mice resulted in a reduction of gonadal fat mass (Table 1) (Windahl S. H. et al (1999) supra; Daci E. et al (2000) supra; Turner R. T., et al (1994) Endocr Rev, 15, 275; Turner R. T., (1999) supra; Bucher N. L. (1991) J Gastroenterol Hepalol, 6, 615; Clarke A. G. & Kendall M. D. (1994) supra; Couse J. F. & Korach K. S. (1999) supra).

TABLE 2
Effects of Estrogen on Fat Levels

| | | Effect of Estrogen (%) | | ERα/β | |
|---|---|---|---|---|---|
| Parameter | | WT | DERKO | Dependent | Independent |
| Fat | Weight | −29.8 ± 333** | −2.0 ± 5.2++ | 93% | 7% |

In Table 2, the left part describes the effects of estrogen on fat in ovx wild type (WT) and DERKO mice. Three months old ovx mice were treated for three weeks with 2.3 ag/mouse/day of 17β-estradiol 5 days/week or olive oil as control (=vehicle). n=7 for WT vehicle, n=7 for WT estrogen, n=7 for DERKO vehicle, n=8 for DERKO estrogen. Values are given as means SEM and expressed as % increase over vehicle treated animal. **=p<0.01 compared with vehicle treated mice. ++=p<0.01 effect of estrogen in DERKO compared with the effect of estrogen in WT, Student t-test. The right part of Table 2 describes the calculation of estrogen receptor α/β dependent and independent effects of estrogen. The effects of estrogen in WT and DERKO mice, as described in the left part of the table, were used for the calculation of the proportion of ERα/β dependent and independent effects of estrogen. The values are given as % of the total effect seen in WT mice.

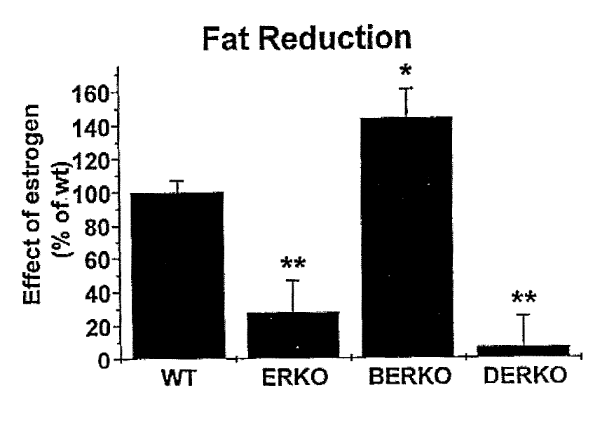

In the present invention, the gonadal fat mass was reduced by estrogen in WT and BERKO mice, but not in ERKO or DERKO mice, demonstrating that ERα is responsible for this effect (FIG. 6). The estrogen hyperresponsiveness in BERKO mice, regarding fat reduction (FIG. 6) may be the result of an unopposed ERα activity.

While the invention has been described in combination with embodiments thereof, it is evident that many alternatives, modifications and variations will be apparent to those skilled in the art in light of the foregoing description. Accordingly, it is intended to embrace all such alternatives, modifications and variations as fall within the spirit and broad scope of the appended claims. All patent applications, patents, and other publications cited herein are incorporated by reference in their entireties.

What is claimed is:

1. A method of treating or preventing obesity in a mammalian subject, comprising the step of supplying an ERα selective compound to said mammalian subject.

2. The method of claim 1, wherein said ERα selective compound is an ERα agonist.

3. The method of claim 1, wherein said mammalian subject is adult.

4. The method of claim 1, wherein said mammalian subject is human.

5. The method of claim 1, wherein gonadal fat levels are reduced as a percentage of body weight to about 1.25% or below.

6. A pharmaceutical composition for the treatment or prevention of obesity, comprising an ERα selective compound and a pharmaceutically acceptable carrier.

7. The pharmaceutical composition of claim 6, wherein said ERα selective compound is an ERα agonist.

8. A method of screening compounds for efficiacy in the treatment or prevention of obesity, comprising the step of determining the ER binding properties of said compounds.

9. The method of claim 8, wherein said compounds are selected on the basis of being ERα selective compounds.

10. The method of claim 9, wherein said compounds which are selected are ERα selective agonists.

11. A method of reduction of serum lipoprotein levels in a mammalian subject, comprising the step of supplying an ERα selective compound to said subject.

12. The method of claim 11, wherein said ERα selective compound is an ERα agonist.

13. The method of claim 11, wherein said subject is adult.

14. The method of claim 11, wherein said subject is human.

15. A pharmaceutical composition for the reduction of serum lipoprotein levels, comprising an ERα selective compound and a pharmaceutically acceptable carrier.

16. The pharmaceutical composition of claim 15, wherein said ERα selective compound is an ERα agonist.

17. A method of screening compounds for efficiacy in the reduction of serum lipoprotein levels, comprising the step of determining the ER binding properties of said compounds.

18. The method of claim 17, wherein said compounds are selected on the basis of having ERα agonist activity.

19. The method of claim 17, wherein said compounds which are selected are ERα selective agonists.

20. A method of screening compounds for use in the treatment of obesity and/or the reduction or lowering of serum lipid levels, the method comprising the use of cells, tissues in which an ER has been disrupted and selecting compounds which are ERα agonists.

21. The method of claim 20, wherein whole animals are used.
